(19) United States
(12) Patent Application Publication
(10) Pub. No.: US 2012/0232448 A1
(43) Pub. Date: Sep. 13, 2012

(54) NEW KINESIOLOGY TAPE

(76) Inventor: Dirk WÜST, Schopfheim (DE)

(21) Appl. No.: 13/476,149

(22) Filed: May 21, 2012

(30) Foreign Application Priority Data
Dec. 23, 2010 (EP) EP 10016029.0

(51) Int. Cl. A61F 5/00

(52) U.S. Cl. 602/1

(57) ABSTRACT

The invention relates to a dermal body-adhesive tape, comprising an elastic woven fabric and an adhesive coating layer applied thereon, characterized in that the adhesive coating layer is covered with a plurality of fascial bodies.

The present invention relates to a dermal pressure-sensitive and elastic body-adhesive tape, especially a new Kinesiology Tape.

The original Kinesio® tape was developed in the early 1970's by Kenzo Kase a Japanese chiropractor. The Kinesio®-Tape is highly elastic, breathable, water resistant and skin friendly. The Kinesio®-Tape is like a second layer of skin and is designed to expand and contract along with the skin without restricting freedom of movement. The tape is supporting the natural movement, it is pain killing and stimulates metabolic processes.

The Kinesio® tape is made of 100% cotton and has an acrylic adhesive coating layer applied thereon. The tape is self adhesive due to the acrylic layer. The air- and moisture permeable Kinesio-Tape is designed to be the same thickness and elasticity of the dermis. The tape is stretchable in all directions. The term “Kinesio®-tape” is well known to those skilled in the art. The tape is commercially available, e.g by Bivax GmbH, Dortmund.

The elasticity of the Kinesio®-Tape of 130 to 140% is comparable with the elasticity of human muscles. This specific elasticity combined with a specific taping techniques allows the normalization of muscular function and the promotion of the flow of lymphatic fluids and blood.

The Kinesio® Tape has a wave-like acrylic adhesive pattern with spaces between the waves to allow the skin to breathe. The tape is thus air- and moisture permeable. Once applied, body heat activates the adhesive property.

U.S. Pat. No. 5,861,348 (Kinesio. Corp) describes a Kinesio® Tape. In FIG. 1 there is shown the pressure-sensitive adhesive layers of the tape. The adhesive layer pattern is wave shaped parallel strips of adhesive covered cloth alternating with parallel shaped strips of exposed cloth.

Despite the successful application of the Kinesiology Tape and its unlimited potential to aid in rehabilitating and avoiding muscle injuries, there is a need to further develop the classical Kinesio® Tape in order to optimize and improve its effect.

It has been found that a combination of the Kinesiology Tape with fascial bodies substantially increases the effect of the Kinesio® tape.

Thus, the present invention relates to a dermal body-adhesive tape, comprising an elastic woven fabric and an adhesive coating layer applied thereon, characterized in that the adhesive coating layer is covered with a plurality of fascial bodies. (claim 1)

The elastic woven fabric can be stretched in all directions. The stretch capability is 120-150%; e.g. 130-140%.

The woven fabric is preferably made of cotton (claim 2). Other materials commonly used for bandages may also be used such as nonwovens, cellulose, polyester, polyamide, acetate and blended fabrics. The woven fabric may be colored and the inventive body-adhesive tape is thus available in all colors.

The adhesive coating should not irritate the skin. The adhesive coating is preferably an acrylic coating (claim 3). Other adhesives commonly used for bandages may also be used such as e.g. rubber adhesive.

The adhesive coating covers the woven fabric. Preferably, the woven fabric is not covered completely, parallel strips of adhesive covered fabric are alternating with parallel shaped strips of uncovered fabric. The adhesive coating layer may have differed shapes, it can be e.g. wave shaped or stripe shaped, preferably wave shaped.

The tape, with the adhesive coating layer, is placed on a protective paper backing in order to protect the adhesive coating.

In a preferred embodiment the dermal body-adhesive tape (layer 1 and 2) is a Kinesiology-tape. (claim 4)

The woven fabric of the Kinesio® tape is made of cotton. The adhesive coating layer is an acrylic coating layer which is wave shaped. Wave shaped uncovered cotton is in between the wave shaped acrylic coating.

In one embodiment the fascial bodies are small particles. (claim 5) The particles may have any shape, e.g. sphere, ellipsoid, polyeder (e.g. tetraeder) cube, pyramid, cone, prism, etc. The particles on the adhesive layer may be unishaped or may have different shapes. FIG. 1 shows an inventive tape having spherical particles.

Length, width and height of the fascial particles are variable. The height is, for example, 0.1-20 mm or 0.1-15 mm, preferably 0.5-5 mm. Length and width are, for example, between 1-10 mm. The data are standard values. The fascial particles may also be larger.

The distribution of the fascial particles on the adhesive coating layer is even or uneven. An example for an even arrangement is the arrangement of the fascial particles in rows.

Preferably the fascial particles are equally distributed throughout the whole adhesive coating layer (claim 6). It is also possible that the fascial particles are only partly distributed throughout the adhesive coating layer. The number of fascial particles is variable and is e.g. 50-200 particle/100 cm².

In another embodiment the fascial bodies are ribbon shaped (claim 7). The fascial ribbons may be arranged on the adhesive coating layer in rows or netlike. Length, width and height of the fascial ribbons are also variable. The height is, for example, 0.1-20 mm or 0.1-15 mm, preferably 0.5-5 mm.

The fascial bodies are made of any material which is skin-friendly, easily formable to particles or ribbons and which is easy to glue. The fascial bodies are, for example, made of skin-friendly plastic (claim 8). Skin friendly plastic is, for example, expanded polystyrene, better known as Styropor®, rubber, thermoplastics, silicone, polyolefines, etc. The fascial particles are, for example made of Styropor® (claim 9).

The fascial bodies may also be made of natural materials such as wood, sand or cotton.

The fascial bodies must not be too hard so that no unpleasant pressure points develop.

Important is that the fascial bodies adhere well to the adhesive coating layer 2.

The surface of the fascial bodies (particles and ribbons) may also have an adhesive layer applied thereon. Due to said additional adhesive layer the adhesion of the inventive tape on the skin is improved.

The fascial bodies (particles and ribbons) may also have different colors. Also the adhesive coating layer may be colored.

In one embodiment the inventive tape has an alkaline pH value. (claim 10)

The alkaline pH value is reached by immersing the inventive tape into an alkaline solution, or spraying or otherwise applying an alkaline solution such as a NaHCO₃ solution followed by drying the tape.

An inventive tape having an alkaline pH value has an additional advantage namely that the connective tissue can be better deacidified through the skin. The pH value is e.g. in the range of 8-10.

When using fascial bodies having a wettable surface such as bodies made of methacrylates and their copolymers or made of natural materials, it is also possible to reach an alkaline pH value by immersing the bodies into the alkaline solution or spraying or otherwise applying an alkaline solution on the fascial bodies.

An alkaline solution may promote additional dissolution of minerals. An inventive tape may thus be obtained containing minerals (claim 11).

The dissolved minerals support the expulsion of toxins and waste products through the skin, Suitable minerals are those contained in commercial available mineral powders such as, for example, potassium, -calcium, -magnesium salts.

The inventive tape is used as orthopaedic bandage (claim 12).

The inventive tape is used to manufacture a tape bandage to be used for therapeutic applications, especially for the treatment of joint injuries and sports injuries (claims 13 and 14).

The invention further relates to a method for treatment of joint injuries and of sports injuries comprising contacting the injured area with the inventive dermal body-adhesive tape (claim 15).

### DESCRIPTION OF THE FIGURES

FIG. 1 illustrates the schematic structure of the inventive tape.

The adhesive coating layer 2 is applied on a stretchable woven fabric 1. The fascial bodies (particles) 3 are applied on the adhesive coating layer. The fascial particles 3 adhere firmly to the coating layer 2, in such a manner that they cannot slip or shift when the tape bandage is put in place. A protective layer 4, preferably a paper layer, is only present before using the tape in order to protect the coating layer.

FIG. 1a is a plan view showing the inventive tape. The spheric fascial particles 3 are equally distributed throughout the whole surface of the adhesive coating layer 2.

When using a Kinesio® tape as body-adhesive tape, the adhesive coating layer 2 is wave shaped. The uncoated wave pattern 5 is also wave shaped. The fascial particles are either completely within the area of the adhesive coating layer or they extend into the area of the uncoated woven fabric 5.

FIG. 1b is a cross section showing the inventive tape. The woven fabric 1 is coated with the adhesive coating layer 2. There are also uncoated areas 5 of the woven fabric. The fascial particles 3 are on the adhesive layer. The protective layer 4 is applied on top.

FIG. 2 is a cross section and a plan view showing the inventive tape. The fascial bodies 3 are ribbons which are arranged like a net.

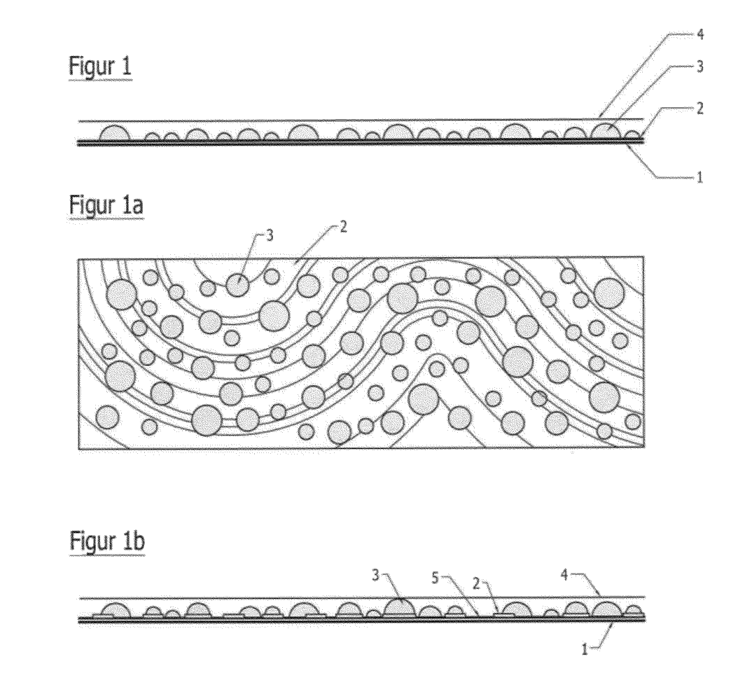

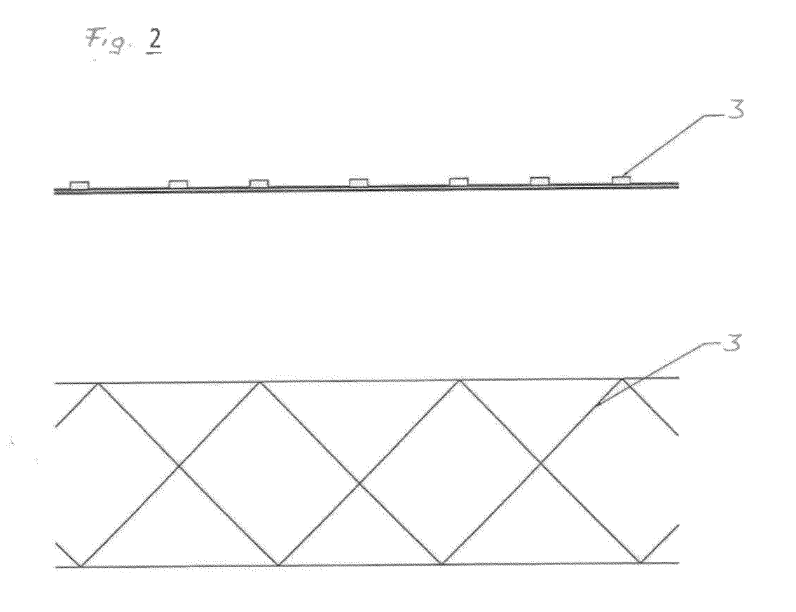

The classical Kinesio® Tape works, amongst others, due to forming skin folds.

The skin is stretched when the tape is applied. Returning back to the unstretched position, the skin together with the glued tape forms wave shaped skin folds. On skin, the Kinesio® Tape pulls the upper layers of skin, creating more space between the dermis and lower dermis. The space created is believed to improve lymphatic flow. Irritation of the skin receptors is decreased. The bodies power of self healing is supported.

### ADVANTAGES OF THE INVENTIVE TAPE

The fascial bodies additionally apply a slight pressure on the skin. When moving said additional pressure increases the massage effect of the known Kinesio® Tape in the lower dermis, whereby the following parts of the body are positively supported:

Skin sensors and pain receptors

proprioceptors

Muscles and insertions of the muscles, fascial tissue,

Ligaments and capsules

Lymphatic system and circulatory system

Meridians and acupuncture points

Scar tissue

Cranio sacral tissue

Visceral tissue

Due to the fascial bodies the inventive tape has a relaxing, soothing and increasing metabolic effect. The efficacy of these effects, which are also assigned to the Kinesio® tape, is improved.

The proper taping technique of the inventive tape is essential to achieve optimum adhesion and efficacy when using the Kinesio® tape

There are different taping techniques depending on the situation
1. The affected skin area is slightly stretched. The tape is applied unstretched.
2. If the skin part cannot be stretched before applying the tape, the tape should be applied with slight stretch with each end (circa 3 cm) being anchored with no stretch.
3. In painful conditions whereby the pain can be precisely localized, the tape should be applied with slight stretch. It is important to have an unstretched tape end (circa 3 cm) on each end.
4. To improve the adhesion, the edges of the tape are rounded using a scissor.
5. The tape should be applied 30-60 min before a workout or competition.
6. It is possible to spray the ends of the tape with liquid plaster spray or with chloroethyl-spray and allow to dry.

1. A dermal body-adhesive tape, comprising an elastic woven fabric and an adhesive coating layer applied thereon, characterized in that the adhesive coating layer is covered with a plurality of fascial bodies.

2. A dermal body-adhesive tape according to claim 1, wherein the woven fabric is made of cotton.

3. A dermal body-adhesive tape according to claim 1, wherein the adhesive coating is an acrylic coating.

4. A dermal body-adhesive tape according to claim 1, wherein the tape is a Kinesiology tape.

5. A dermal body-adhesive tape according to claim 1, wherein the facial bodies are particles.

6. A dermal body-adhesive tape according to claim 5, wherein the particles are equally distributed throughout the whole adhesive coating layer.

7. A dermal body-adhesive tape according to claim 1, wherein the facial bodies are ribbon shaped.

8. A dermal body-adhesive tape according to claim 1, wherein the facial bodies are made of skin-friendly plastic.

9. A dermal body-adhesive tape according to claim 8, wherein the facial bodies are made of Styropor®.

10. A dermal body-adhesive tape according to claim 1, wherein the tape has an alkaline pH value.

11. A dermal body-adhesive tape according to claim 10, wherein the tape contains minerals.

12. A dermal body-adhesive tape according to claim 1 for use as orthopaedic bandage.

13. A dermal body-adhesive tape according to claim 1 for use as tape bandage to be used for therapeutic applications.

14. A dermal body-adhesive tape according to claim 13 for use in the treatment of joint injuries and sports injuries.

15. A method for treatment of joint injuries and of sports injuries comprising contacting the injured area with a dermal body-adhesive tape according to claim 1
